(19) United States
(12) Patent Application Publication
(10) Pub. No.: US 2007/0009455 A1
(43) Pub. Date: Jan. 11, 2007

(54) Whitening and antionxidative cosmetic composition containing resveratrol and method for preparing the same

(76) Inventors: Hyo-Jung Kim, Seoul (KR); Sung-won Jung, Incheon (KR); Hyuk Kim, Siheung-si (KR); Suk-jae Kim, Seoul (KR); Joo-dong Lee, Suwon-si (KR); Hee-chang Ryoo, Seoul (KR); Ae-young Kwon, Incheon (KR); Mi-ra Jo, Incheon (KR)

Correspondence Address: FISH & RICHARDSON PC, P.O. BOX 1022, MINNEAPOLIS, MN, 55440-1022, US

(21) Appl. No.: 10/558,348

(22) PCT Filed: May 27, 2004
(86) PCT No.: PCT/KR04/01247

§ 371 Date: Nov. 23, 2005

(30) Foreign Application Priority Data
May 26, 2003 (KR) 10-2003-0033356
Oct. 27, 2003 (KR) 10-2003-0075004

(51) Int. Cl. A61K 8/97; A61K 8/64; A61K 8/73; A61K 8/49

(52) U.S. Cl. 424/62; 424/70.14; 424/74; 424/70.13

(57) ABSTRACT

Provided are a stabilized, skin-whitening and antioxidative cosmetic composition including resveratrol as a main component and either a primary stabilizer selected from cyclodextrin, polyethyleneglycol, and a mixture thereof, or a combination of the primary stabilizer and a secondary stabilizer selected from an alpha-lipoic acid, a water-soluble whitening composition, a phellodendron extract, an alteromonas ferment extract, and a mixture thereof, and a method for preparing the same. The cosmetic composition provides excellent skin whitening and antioxidative effects for a long-term even when used in a small quantity. Furthermore, solubility of the resveratrol and the alpha-lipoic acid in polyol or ethanol is enhanced, thereby improving low use satisfaction and skin irritation that may be caused due to excess use of an organic solvent. In addition, problems such as crystal precipitation at low temperature, off-flavor and discoloration upon exposure to sun light can be solved.

### TECHNICAL FIELD

The present invention relates to a whitening and antioxidative cosmetic composition containing resveratrol and a method for preparing the same. More particularly, the present invention relates to a cosmetic composition containing resveratrol as a main component and either a primary stabilizer selected from cyclodextrin, polyethyleneglycol, and a mixture thereof, or a combination of the primary stabilizer and a secondary stabilizer selected from an alpha-lipoic acid, a water-soluble whitening composition, a phellodendron extract, an alteromonas ferment extract, and a mixture thereof, and a method for preparing the same.

### BACKGROUND ART

The skin is a very important organ that protects the internal organs of the human body and effects biochemical and physical functions while being in direct contact with external environment. It is largely composed of three different layers, i.e., epidermis, dermis, and subcutaneous tissue. Skin color in humans is mainly determined by number, size, type, and distribution of melanosomes containing melanin that are spread throughout skin cells. Melanosomes are produced by melanin cells. Melanin is a dark pigment which is produced in melanocytes of epidermis.

Melanin absorbs the energy of UV light irradiated by sun light and prevents the light from penetrating into deeper skin cells. However, when melanin production is abnormally low, a skin disease such as leukoderma may be caused. On the other hand, overproduction of melanin by UV exposure and the like may cause a skin damage such as freckles and lentigo-type freckles and may contribute to development of a skin cancer.

Resveratrol is present in a wide variety of gymnosperms and angiosperms and is mainly synthesized in the form of glycoside linked with sugar group even though some of resveratrol is in free state [Gorham, J., Prog. Phytochem., 6:203-209, 1980]. Resveratrol exists in cis and trans isoforms. Stable trans-resveratrol is common in nature [Trela, B. C. & Waterhouse, A, L., J. Agric. Food Chem., 44(5): 1253-1257, 1996].

Resveratrol is mainly found in the shell, seed, stem, and leaf of grapes. In particular, resveratrol is abundantly contained in grapes such as Vitis vinifeera, Vitis rotundifolia, and Vitis labrusca. Resveratrol is also found in wines made from these grapes, and extracts and powders of the aforementioned natural sources. In addition, resveratrolis contained in roots of Polygonum sp. such as Polygonum cuspidatum and Polygonum multiflorum, belonging to the Polygonaceae family.

As used herein, the term “resveratrol” covers trans-3,4,5-trihydroxystilbene (i.e., suitable resveratrol) as represented by the following Formula 1 and its cis-isomers, glycosides thereof, and resveratrol-containing extracts and powders obtained from a suitable plant.

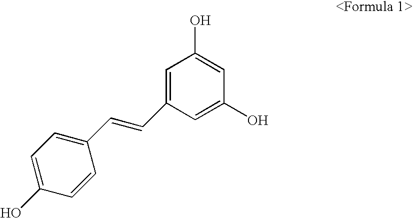

Trans-resveratrol has been reported as a physiologically active substance that has an anticancer effect (Jang M., et al., Sci., 275:218-220, 1997), antithrombotic effect (Chung, M. L. et al., Planta Med., 58:274-276, 1992; Frankel, E. N. et al., Lancet, 341:1103-1104, 1993; Bertrilli, A. A. E. et al., Int. J. Tissue React., 17:1-3, 1995; Pae-Asciak, C. R. et al., Clin. Chim. Acta., 235:207-219, 1995), anti inflammatory effect (Kimura, Y. et al., Biochim. Biophys. Acta., 834:275-278, 1985), and antihyperlipidemic effect (Arichi, H., et al, chem. Pharm. Bull., 30(5):1766-1770, 1982).

In particular, recent studies have shown that trans-resveratrol is a phytoestrogen that produces estrogen-like effects, and thus, there has been an increasing interest in trans-resveratrol as phytoestrogen (Gehm, B. D. et al., Proc. Nasl. Acad. Sci., 94:14138-14143, 1997). A cosmetic composition containing a grape extract is disclosed in Japanese Patent No. 06336421, U.S. Pat. Nos. 5,683,683, 5,439,672, and 5,171,577. PCT/EP98/04223 discloses a cosmetic composition containing resveratrol. According to the disclosure in PCT/EP98/04223, the cosmetic composition prevents proliferation and differentiation of keratinocytes or skin irritation by alpha-hydroxy acid. However, this patent document is silent about solutions to problems such as low use satisfaction, skin irritation, and crystal precipitation at low temperature that may be caused by excess use of polyol or ethanol that is used as a solvent in a cosmetic product due to low solubility of resveratrol in the polyol or the ethanol, and off-flavor and discoloration upon exposure to sun light.

In spite of excellent whitening and antioxidative effects, there is a problem in that resveratrol may be precipitated as a crystal when used in a cosmetic composition containing water. For this reason, excess ethanol or polyol must be used. That is, 0.1 wt % of resveratrol requires use of 10 wt % of an organic solvent such as polyol, which makes it difficult to use a high content of resveratrol. A high content of resveratrol can be used only in a substantially water-free cosmetic composition. However, a water-free cosmetic composition using only an organic solvent such as ethanol or polyol is hardly commercially available due to severe skin irritation and low use satisfaction. In particular, vitamin C that is difficult to be stable in a cosmetic composition is prepared as a “polyol in silicone” or “silicone in polyol” system. However, this formulation system has low use satisfaction, complex preparation process, and expensive material cost, and thus, is limitedly used in a cosmetic product.

Meanwhile, alpha-lipoic acid as used herein has strong antioxidative activity. It is known that alpha-lipoic acid serves to facilitate production of glutathione that is another antioxidative substance of the human body or help functions and regeneration of vitamin E and C, thereby increasing an antioxidative effect in the human body.

Based on these effects of alpha-lipoic acid, alpha-lipoic acid is currently used in the treatment of diabetes, nervous disease, cataract, heart failure, atherosclerosis, and the like. In addition, alpha-lipoic acid is known to have excellent moisturizing capability by ceramide production (U.S. Pat. No. 5,472,698A), therapeutic effects of skin inflammation, necrosis, tumor, allergy, and the like (Germany Patent No. 441,708A), therapeutic effects of allergy, inflammation, and tanning (WO 9508564A), skin whitening and tyrosinase inhibitory activity (Japanese Patent No. 63008315A), and acne prevention and relief by maintenance of homeostasis (Korean Patent Laid-Open Publication No. 1999-025848). However, there may arise problems such as low use satisfaction by excess use of polyol in a cosmetic product due to low solubility of alpha-lipoic acid in the polyol, unique odor due to a sulfur element, and off-flavor and discoloration due to separation of thiol group upon exposure to light.

Therefore, the present inventors have developed a whitening and antioxidative cosmetic composition which stably contains resveratrol that had been difficult to be used in a cosmetic product, and thus, has no side effects such as skin irritation.

In view of the above problems, the present invention provides a cosmetic composition containing resveratrol which is excellent in whitening and antioxidative effects and a method for preparing the same.

The above and other objects of the present invention can be accomplished by embodiments of the present invention as will be described hereinafter.

### DISCLOSURE OF THE INVENTION

Therefore, according to an aspect of the present invention, there is provided a whitening and antioxidative cosmetic composition including resveratrol.

The resveratrol may be used in an amount of 0.001-10.0 wt %, based on the total weight of the cosmetic composition.

The cosmetic composition may further include a primary stabilizer selected from the group consisting of cyclodextrin, polyethyleneglycol, and a mixture thereof.

The primary stabilizer may be used in an amount of 0.1 to 50 wt %, based on the total weight of the cosmetic composition.

The cosmetic composition including the resveratrol and the primary stabilizer may further include a secondary stabilizer selected from the group consisting of an alpha lipoic acid, a water-soluble whitening composition, a phellodendron extract, an alteromonas ferment extract, and a mixture thereof.

The secondary stabilizer may be used in an amount of 0.01 to15.0 wt %, basedonthe total weight of the cosmetic composition.

The water-soluble whitening composition may be selected from the group consisting of a citrus unshiu peel extract, an aminoethylphosphinic acid, Waltheria indica extract, Guava Phenone, an aqueous hydrolyzed yeast solution, and a mixture thereof.

According to another aspect of the present invention, there is provided a method for preparing a whitening and antioxidative cosmetic composition, including: (a) mixing a solvent and a primary stabilizer; (b) adding resveratrol to the mixture of step (a); and (c) adding a secondary stabilizer selected from the group consisting of an alpha-lipoic acid, a water-soluble whitening composition, a phellodendron extract, an alteromonas ferment extract, and a mixture thereof, to the mixture of step (b).

Hereinafter, the present invention will be described in detail.

The present invention provides a whitening and antioxidative cosmetic composition. More particularly, the present invention provides a cosmetic composition including a stabilized resveratrol and a primary stabilizer selected from cyclodextrin, polyethyleneglycol, and a mixture thereof. Preferably, the cyclodextrin is hydroxypropyl beta cyclodextrin and the polyethyleneglycol is polyethyleneglycol 400 or polyethyleneglycol 32.

The cosmetic composition including the resveratrol and the primary stabilizer may further include a secondary stabilizer selected from the group consisting of an alpha-lipoic acid, a water-soluble whitening composition, a phellodendron extract, an alteromonas ferment extract, and a mixture thereof.

The water-soluble whitening composition (Whitegenic, Korean Patent Application No. 10-2002-0009503) may be selected from the group consisting of a citrus unshiu peel extract, an aminoethylphosphinic acid, Waltheria indica extract, Guava Phenone, an aqueous hydrolyzed yeast solution, and a mixture thereof.

In more detail, in the cosmetic composition including the stabilized resveratrol, the resveratrol maybe used in an amount of 0.001-10.0 wt %, preferably 0.1-5.0 wt %, based on the total weight of the cosmetic composition. The primary stabilizer may be used in an amount of 0.1-50 wt %, preferably 1.0-20.0 wt %, based on the total weight of the cosmetic composition. The secondary stabilizer may be used in an amount of 0.01-15.0 wt %, preferably 0.1-10.0 wt %, based on the total weightof thecosmetic composition.

In the cosmetic composition including the stabilized resveratrol, the balance except the resveratrol, the primary stabilizer, and the secondary stabilizer is a solvent selected from polyol, ethanol, and water.

The alpha-lipoic acid with antioxidiative activity as used herein is represented by the following Formula 2. The alpha-lipoic acid is a co-factor that is essential in energy metabolism or cellular respiration of organism including microorganisms and humans, together with thiotic acid, thioctan, and 1.2-dithiolane-3-pentanoic acid. The alpha-lipoicacid has been recognized as a strong antioxidant since it was first isolated in 1950.

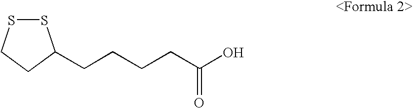

The alpha-lipoic acid is soluble in ethanol or polyol which is generally used in a cosmetic composition. However, the alpha-lipoic acid has a disadvantage in that it is quickly converted to dihydrolipoic acid represented by the following Formula 3, in a solution, thereby generating an odor.

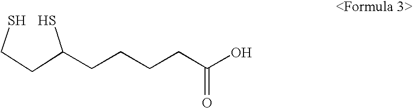

According to the cosmetic composition including the stabilized resveratrol, the resveratrol is used in an amount of 0.001-10.0 wt %. If the content of the resveratrol is less than 0.001 wt %, a desired antioxidative effect may not be obtained. On the other hand, if the content of the resveratrol exceeds 10.0 wt %, an antioxidative effect relative to an increased material cost may be insignificant.

A solvent as used herein may be a solvent commonly used in the cosmetic industry, for example ethanol, 1,3-butyleneglycol, propyleneglycol, dipropyleneglycol, pentyleneglycol, or glycerine. Ethanol is irritating and generates a specific smell when used in a high content. Therefore, it is preferable to use ethanol in an amount of about 10 wt % or less. In this regard, ethanol is not used in a cosmetic composition for a sensitive skin. A cosmetic composition including a high content of polyol may cause sticky skin feel, thereby lowering use satisfaction. Therefore, it is preferable to use polyol in an amount of 10 wt % or less.

The phellodendron extract that is used herein as a stabilizer is an extract obtained by extracting Phellodendron plant with ethanol or 1,3-butyleneglycol. The bark of Phellodendron amurense Ruprecht or Rutaceae is used. The phellodendron plant is cold, bitter, and non-toxic. It is effective in treatment of fever and jaundice that are concentrated in the five viscera and the stomach and intestines, diarrhea and dysentery, gynecopathy, scabies and scabs, eye fever and ache, sore throat, and a high fever that can melt bone. In addition, it is well known that the phellodendron plant has antifungal, antiinflammatory, and antivirus effects. However, there have been no reports that the phellodendron plant can prevent discoloration and off-flavor by exposure to UV light. The present inventors found that the phellodendron plant can prevent phase instability of effective components by sun light and the like. The phellodendron extract is commercially available under the trade name of Phellodendron Bark BG (Koei, Japan).

The alteromonas ferment extract that is used herein as another stabilizer is deepsane composed of 11 glucosides that is a fermentation production of Alteromonas macleodii that is a microorganism present in a severe environment having seawater depth of 3,000 m or atmospheric pressure of 300 bar. The present inventors found that the alteromonas ferment extract has a molecular weight of 1.8×10⁶ Dalton and a skin irritation relief effect. The alteromonas ferment extract is commercially available under the trade name of Abyssine 657 (Lanatech, France).

In the present invention, the secondary stabilizer selected from the water-soluble whitening composition, the alpha-lipoic acid, the phellodendron extract, the alteromonas ferment extract, and the mixture thereof may be used in an amount of 0.01-15 wt %, based on the total weight of the cosmetic composition. If the content of the secondary stabilizer exceeds 15 wt %, an ef fect enhancement relative to an increased material cost may be insignificant.

The cyclodextrin and the polyethyleneglycol used as the primary stabilizer in the cosmetic composition including the resveratrol may be represented by the following Formulae 4a and 4b, respectively. Preferably, the cyclodextrin is hydroxypropyl beta cyclodextrin and the polyethyleneglycol is polyethyleneglycol 400 or polyethyleneglycol 32. 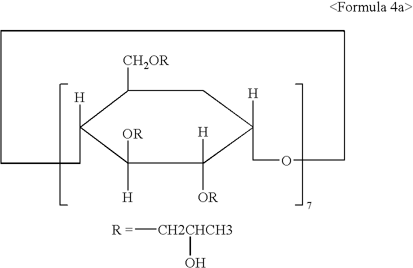 H(OCH₂CH₂)nOH
n=32, 400 <Formula 4b>

The primary stabilizer must be used in an amount of 5-20 wt %, based on the total weight of effective components or in an amount of 0.1-50 wt %, based on the total weight of the cosmetic composition including the stabilized resveratrol. If the content of the primary stabilizer exceeds 20 wt %, based on the total weight of the effective components, a cost increase and use satisfaction lowering by excess use of the primary stabilizer may be caused.

The cosmetic composition including the stabilized resveratrol of the present invention can solve phase instability with time such as discoloration, off-flavor, and crystal precipitation that may be caused during preparation or storage and reduce skin irrigation, simultaneously with exhibiting continuously an excellent antioxidative activity.

A method for preparing the cosmetic composition including the stabilized resveratrol according to the present invention includes:

(a) mixing a solvent and a primary stabilizer;

(b) adding the resveratrol to the mixture of step (a); and

(c) adding a secondary stabilizer selected from a water-soluble whitening composition, an alpha-lipoic acid, a phellodendron extract, an alteromonas extract, and a mixture thereof, to the mixture of step (b).

In the method for preparing the cosmetic composition including the resveratrol, the solvent may be polyol, ethanol, or water.

In step (a), the primary stabilizer may be cyclodextrin or polyethyleneglycol, and preferably, hydroxypropyl beta cyclodextrin, polyethyleneglycol 32, polyethyleneglycol 400, or a mixture thereof.

The method for preparing the cosmetic composition including the resveratrol is generally carried out at room temperature and under atmospheric pressure. If necessary, heating may be used.

In the cosmetic composition including the resveratrol, the primary stabilizer is used in an amount of 1.0-50.0 wt %, based on the total weight of the cosmetic composition.

A cosmetic composition of the present invention may be formulated as all formulations that can be prepared by solubilization, emulsification, or dispersion, for example toner, lotion, cream, essence, sunscreen cream, cleansing foam, cleansing cream, or pack, but are not limited thereto.

### BEST MODE FOR CARRYING OUT THE INVENTION

To solve problems such as low solubility of resveratrol that restricts its utility in a cosmetic composition in spite of excellent antioxidative effect and the like, and discoloration, off-flavor, and crystal precipitation during long-term storage, a cosmetic composition of the present invention includes resveratrol stabilized by a primary stabilizer or a combination of the primary stabilizer and a secondary stabilizer. In Experimental Examples as will be described hereinafter, to evaluate long-term effects with time of the cosmetic composition, a long-term effect of an antioxidative activity is evaluated by measuring radical scavenging ability. In addition, skin safety is evaluated by skin irritation test based on patch test in skins of healthy adults. Phase stability in the cosmetic composition is evaluated by observing color change and crystal precipitation with time after exposure to 40° C., 4° C., and sun light.

The whitening activity of the cosmetic composition including the resveratrol is evaluated by measuring inhibitory activity against tyrosinase that contributes to melanin biosynthesis, radical scavenging activity, and antiproliferative activity against melanoma cells that have a similar condition to living system.

According to these test results, the cosmetic composition including the resveratrol and either the primary stabilizer or a combination of the primary stabilizer and the secondary stabilizer of the present invention exhibits an excellent whitening activity, phase stability, and skin safety, relative to a commercially available whitening agent such as albutin and cojic acid, and thus, can be efficiently used as a whitening cosmetic composition.

Hereinafter, the present invention will be described more specifically by Examples but the present invention is not limited to or by them.

### EXPERIMENTAL EXAMPLE 1

### Tyrosinase Inhibitory Activity Test

Inhibitory activity of resveratrol against tyrosinase that participates in main processes of melanin biosynthesis was measured to evaluate a whitening effect of resveratrol and the result is presented in Table 1 below.

Tyrosine used as a substrate in main processes of melanin biosynthesis, mushroom tyrosinase, or tyrosinase fragments separated from melanocytes was added to test samples and incubated for a predetermined time. The absorbance of dopachrome that was a reaction product was measured at an absorption wavelength to evaluate the whitening effects of the test samples.
Tyrosinase inhibitory activity (%)=100−((ODₑₓₚ−OD_{con})/OD_{std}×100)

ODₑₓₚ: Absorbance of test sample containing tyrosinase

OD_{con}: Absorbance of test sample containing no tyrosinase

OD_{std}: Absorbance of standard sample containing tyrosinase

TABLE 1
Test results of tyrosinase inhibitory activity

| Sample | *IC₅₀ (μg/ml) |
|---|---|
| Resveratrol | 2 |
| Cojic acid | 5 |
| Available licorice extract | >50 |
| White mulberry powder | >50 |
| Vitamin C | >100 |
| Albutin | >100 |
| Water-soluble whitening | >100 |
| composition (*Whitegenic) | |
| White mulberry extract | >1000 |
| Grapeseed extract | >1000 |

*IC₅₀: 50% tyrosinase activity inhibition concentration (as the value of IC₅₀ decreases, tyrosinase inhibitory activity increases)
*Whitegentic (trade name, Korea):a mixture of Whitegentic 1 (Sederma, France):Whitegenic 2 (Exymol, Monaco):Whitegenic 3 (Sero, France) or Guava Phenone (10% aqueous solution):Whitegenic 4 (Sapporo, Japan) (5:1:0.3:2)

It can be seen from Table 1 that the resveratrol and the cojic acid have excellent tyrosinase inhibitory activity.

### EXPERIMENTAL EXAMPLE 2

### Free Radical Scavenging Activity Test

To evaluate the antioxidative activity of the resveratrol, a free radical scavenging activity test was performed as the follows and the results are presented in Table 2 below.

When methanol solutions of test samples were converted to clear solutions due to scavenging of relatively stable deep blue-colored DPPH(1,1-diphenyl-2-hydrazyl) radicals, the degree of reduction of the absorbance of the methanol solutions was measured at 516 nm.
Free radical scavenging activity (%)=100−( (ODₑₓₚ−OD_{con})/OD_{std}×100)

ODₑₓₚ: Absorbance of test sample containing DPPH

OD_{con}: Absorbance of test sample containing no DPPH

OD_{std}: Absorbance of standard sample containing DPPH

TABLE 2
Test results of free radical scavenging activity

| Sample | IC₅₀ (μg/ml) |
|---|---|
| Vitamin C | 3 |
| Resveratrol | 10 |
| Albutin | >50 |
| White mulberry powder | >100 |
| Cojic acid | >200 |
| Available licorice extract | >250 |
| Grapeseed extract | >250 |
| Water-soluble whitening composition | >250 |
| (*Whitegenic) | |
| White mulberry extract | >1000 |

*IC₅₀: 50% free radical scavenging concentration (as the value of IC₅₀ decreases, free radical scavenging activity increases)
*Whitegenic (trade name, Korea): a mixture of Whitegenic 1 (Sederma, France):Whitegenic 2 (Exymol, Monaco):Whitegenic 3 (Sero, France) or Guava Phenone (10% aqueous solution):Whitegenic 4 (Sapporo, Japan) (5:1:0.3:2)

It can be seen from Table 2 that Vitamin C and resveratrol are effective free radical scavengers.

### EXPERIMENTAL EXAMPLE 3

### Whitening Activity Test Based on Proliferation of Skin Melanocytes

Inhibitory activity of resveratrol against melanin production in melanocytes of healthy persons was measured and the results are presented in Table 3 below.

The whitening activity test based on proliferation of skin melanocytes was performed as the following method:
1. Human skin melanocytes in MGM (Melanocyte Growth Media, Clonetics) media were incubated at a 5% CO₂ incubator at 37° C.
2. A predetermined number of cells (2×10⁶) were cultured in75T culture flasks. When cell confluence was identified, test samples of a predetermined concentration were loaded in the cell cultures and incubated for 2-3 days.
3. The cultures were removed, washed with phosphate buffered saline (PBS), and treated with trypsin solution, to harvest cells.
4. The harvested cells were centrifuged to obtain cell pellets.

The colors of the cell pellets were observed. The cell pellets were dissolved in 1N NaOH and the absorbance was measured at 475 nm.
5. The concentration of melanin was calculated by using absorbance values obtained from melanin standard and then converted to the concentration of melanin/the total protein concentration of melanocytes.
   Antiproliferative activity against melanoma cells(%)=[(A−B)/A]×100

A: concentration of melain/concentration of protein in control

(control: melanocytes normally grown in a culture medium containing no test samples)

B: concentration of melain/concentration of protein in each test sample

TABLE 3
Test results of melanin production inhibitory activity
(concentration of each test sample: 0.02%)

| | Melanin production inhibitory |
|---|---|
| Sample | activity (%) |
| Resveratrol | 53.17 |
| Water-soluble whitening | 35.00 |
| composition (*Whitegenic) | |
| Cojic acid | 22.62 |
| Vitamin C | 19.72 |
| Available licorice extract | 13.20 |
| White mulberry powder | 5.1 |
| Albutin | 2.00 |
| White mulberry extract | 1.80 |
| Grapeseed extract | 1.52 |

*Whitegenic (trade name, Korea): a mixture of Whitegenic 1 (Sederma, France):Whitegenic 2 (Exymol, Monaco):Whitegenic 3 (Sero, France) or Guava Phenone (10% aqueous solution):Whitegenic 4 (Sapporo, Japan) = 5:1:0.3:2

It can be seen from Table 3 that the resveratrol and the water-soluble whitening composition (Whitegenic, Korea) exhibit excellent inhibitory activity of melanin production.

Based on the test results of Experimental Examples, cosmetic compositions capable of sustaining and enhancing the whitening effect of the resveratrol were prepared. To evaluate whether skin whitening activity can be maintained while maintaining excellent skin safety and phase stability, the whitening effect tests of the cosmetic compositions were performed according to the aforementioned melanocytes proliferation test.

### EXAMPLES 1-7 AND COMPARATIVE EXAMPLES 1-8

### Preparation of Highly Concentrated and Stabilized Cosmetic Compositions

To evaluate an enhancement effect of solubility and phase stability of resveratrol, stabilized cosmetic compositions were prepared according to composition ratios presented in Table 4 below (Table 4a for Examples 1-7, Table 4b for Comparative Examples 1-8). Each composition of Examples 1-7 and comparative Examples 1-8 was prepared as the follows.

First, a solvent part including primary stabilizers was sufficiently stirred to obtain a clear solution. Resveratrol used as an effective component was gradually added to the clear solution at room temperature and stirred for 30-60 minutes. After the resveratrol was completely dissolved, components of a secondary stabilizer part were gradually added thereto and stirred for 10-20 minutes to there by obtain a stabilized cosmetic composition.

TABLE 4a
Stabilized cosmetic composition

| Section | Component | Exam. 1 | Exam. 2 | Exam. 3 | Exam. 4 | Exam. 5 | Exam. 6 | Exam. 7 |
|---|---|---|---|---|---|---|---|---|
| Solvent | Purified | To 100 | To 100 | To 100 | To 100 | To 100 | To | To |
| part | water | | | | | | 100 | 100 |
| | Hydroxypropyl | 10.0 | 20.0 | 30.0 | 50.0 | 50.0 | — | — |
| | beta | | | | | | | |
| | cyclodextrin¹⁾ | | | | | | | |
| | Polyethyleneglycol²⁾ | — | — | — | — | — | 20.0 | — |
| | 1,3-butyleneglycol | — | — | — | — | — | — | 5.0 |
| | Ethanol | — | — | — | — | — | — | 5.0 |
| Effective | Resveratrol | 0.5 | — | 2.0 | 5.0 | 5.0 | 2.0 | 0.5 |
| component | | | | | | | | |
| Secondary | Waste-soluble | — | — | — | — | — | — | 20.0 |
| stabilizer | whitening | | | | | | | |
| part | composition | | | | | | | |
| | Alpha-lipoic | — | 2.0 | 3.0 | 5.0 | 5.0 | 5.0 | — |
| | acid | | | | | | | |
| | Phellodendron | 1.0 | 3.0 | 5.0 | 10.0 | 10.0 | 10.0 | — |
| | extract³⁾ | | | | | | | |
| | Alteromonas | — | 1.0 | 3.0 | 5.0 | — | — | — |
| | ferment | | | | | | | |
| | extract⁴⁾ | | | | | | | |

Exam.: Example
¹⁾Celdex HP-beta-CD (Food and Chemical Engineering, Ltd., JAPAN)
²⁾Renex PEG 1500, PEG-400 (Uniqema Americas)
³⁾Pellodendron Bark BG (Koei's, JAPAN)
⁴⁾Abyssine 657 (Lanatech, FRANCE)

TABLE 4b
Stabilized composition

| Section | Component | Comp. 1 | Comp. 2 | Comp. 3 | Comp. 4 | Comp. 5 | Comp. 6 | Comp. 7 | Comp. 8 |
|---|---|---|---|---|---|---|---|---|---|
| Solvent | Purified water | To | To | To | To | To | To | To | To |
| part | | 100 | 100 | 100 | 100 | 100 | 100 | 100 | 100 |
| | Hydroxypropyl beta | 3.0 | 10.0 | — | 50.0 | — | — | — | — |
| | cyclodextrin¹⁾ | | | | | | | | |
| | Polyethyleneglycol²⁾ | — | — | | — | — | — | — | — |
| | 1,3-butyleneglycol | — | — | 30.0 | — | 50.0 | 5.0 | | — |
| | Ethanol | — | — | 10.0 | — | 20.0 | 15.0 | 10.0 | 10.0 |
| Effective | Resveratrol | 1.0 | 2.0 | 3.0 | 5.0 | 5.0 | 0.5 | — | — |
| component | | | | | | | | | |
| Secondary | Water-soluble | — | — | — | — | — | — | — | 20.0 |
| stabilizer | whitening | | | | | | | | |
| part | composition | | | | | | | | |
| | Alpha-lipoic acid | — | | — | 5.0 | 5.0 | — | — | — |
| | Phellodendron | — | — | 3.0 | — | — | — | — | — |
| | extract³⁾ | | | | | | | | |
| | Alteromonas ferment | — | — | 1.0 | — | — | — | — | — |
| | extract⁴⁾ | | | | | | | | |

Comp.: Comparative Example
¹Celdex HP-beta-CD (Food and Chemical Engineering, Ltd., JAPAN)
²⁾Renex PEG 1500, PEG-400 (Uniqema Americas)
³⁾Phellodendron Bark BG (Koei's, JAPAN)
⁴⁾Abyssine 657 (Lanatech, FRANCE)

### EXPERIMENTAL EXAMPLE 4

### Solubility and Phase Stability Test

To compare the solubility and phase stability of resveratrol between the compositions of the present invention and conventional compositions containing a common solvent such as purified water, polyol and ethanol, solubility, flavor, and color were observed immediately after preparation and at predetermined times after preparation and the results are presented in Tables 5 and 6 below.

TABLE 5
Solubility

| | Immediately after | One month after |
|---|---|---|
| Section | preparation | preparation |
| Example 1 | Dissolution | Good |
| Example 2 | Dissolution | Good |
| Example 3 | Dissolution | Good |
| Example 4 | Dissolution | Good |
| Example 6 | | |
| Comparative Example 1 | No dissolution | Crystal precipitation |
| | (presence of crystal) | |
| Comparative Example 2 | Dissolution | Good |
| Comparative Example 3 | Dissolution | Crystal precipitation |
| Comparative Example 4 | Dissolution | Good |
| Comparative Example 5 | Dissolution | Crystal precipitation |

As shown in Table 5, with respect to the compositions of Examples 1-4 and 6 according to the present invention, even when the content of the effective component (resveratrol) was 15 wt %, solubility was good. Even at one month after preparation, crystal precipitation was not observed. As described above, the hydroxypropyl beta cyclodextrin must be used in 5-20 wt %, based on the total weight of the effective component. With respect to the composition of Comparative Example 1 in which the content of the hydroxypropyl beta cyclodextrin was less than 5 wt %, crystals were present due to incomplete encapsulation. On the other hand, if the content of the hydroxypropyl beta cyclodextrin exceeds 20 wt %, an increase of a material cost and lowering of use satisfaction due to excess use of the encapsulating agent may be caused. Therefore, it can be seen that use of a suitable amount of the encapsulating agent is required.

TABLE 6
Phase stability

| Section | | 1 day | 1 month | 2 months | 3 months |
|---|---|---|---|---|---|
| Example 1 | R.T. | ⊚ | ⊚ | ⊚ | ⊚ |
| | 40° C. | ⊚ | ⊚ | ⊚ | 0 |
| | 4° C. | ⊚ | ⊚ | ⊚ | ⊚ |
| | Sun light | ⊚ | ⊚ | 0 | 0 |
| Example 2 | R.T. | ⊚ | ⊚ | ⊚ | ⊚ |
| | 40° C. | ⊚ | ⊚ | 0 | 0 |
| | 4° C. | ⊚ | ⊚ | ⊚ | ⊚ |
| | Sun light | ⊚ | ⊚ | 0 | 0 |
| Example 3 | R.T. | ⊚ | ⊚ | ⊚ | ⊚ |
| | 40° C. | ⊚ | ⊚ | 0 | 0 |
| | 4° C. | ⊚ | ⊚ | ⊚ | ⊚ |
| | Sun light | ⊚ | ⊚ | 0 | 0 |
| Example 4 | R.T. | ⊚ | ⊚ | ⊚ | ⊚ |
| | 40° C. | ⊚ | ⊚ | 0 | 0 |
| | 4° C. | ⊚ | ⊚ | ⊚ | ⊚ |
| | Sun light | ⊚ | ⊚ | 0 | 0 |
| Example 6 | R.T. | ⊚ | ⊚ | ⊚ | ⊚ |
| | 40° C. | ⊚ | ⊚ | 0 | 0 |
| | 4° C. | ⊚ | ⊚ | ⊚ | ⊚ |
| | Sun light | ⊚ | ⊚ | 0 | 0 |
| Comparative | R.T. | ⊚ | ⊚ | ⊚ | 0 |
| Example 2 | 40° C. | ⊚ | 0 | 0 | ▾ |
| | 4° C. | ⊚ | 0 | ▾ | ⋆ |
| | Sun light | 0 | ▾ | ⋆ | ⋆ |
| Comparative | R.T. | ⊚ | ⊚ | 0 | ▾ |
| Example 3 | 40° C. | ⊚ | 0 | ▾ | ⋆ |
| | 4° C. | ⊚ | 0 | ▾ | ⋆ |
| | Sun light | 0 | ▾ | ⋆ | ⋆ |
| Comparative | R.T. | ⊚ | ⊚ | 0 | ▾ |
| Example 4 | 40° C. | ⊚ | 0 | ▾ | ⋆ |
| | 4° C. | ⊚ | 0 | ▾ | ⋆ |
| | Sun light | 0 | ▾ | ⋆ | ⋆ |
| Comparative | R.T. | 0 | ▾ | ⋆ | ⋆ |
| Example 5 | 40° C. | 0 | ▾ | ⋆ | ⋆ |
| | 4° C. | 0 | ▾ | ▾ | ⋆ |
| | Sun light | ▾ | ⋆ | ⋆ | ⋆ |

R.T.: room temperature
⊚ no change,
0: slight discoloration,
▾: remarkable discoloration, slight phase separation, or suspension,
⋆ complete discoloration, phase separation, or precipitation

As shown in Table 6, with respect to the compositions of Comparative Examples 3 and 5 in which hydroxypropyl beta cyclodextrin or polyethyleneglycol was not used as a solvent, crystal precipitation at low temperature occurred (see Comparative Examples 3 and 5) and severe off-flavor by disulfur was generated (see Comparative Example 5). The composition of Comparative Example 4 containing no phellodendron extract and alteromonas ferment extract exhibited severe browning phenomenon upon exposure to sun light, as compared to the composition of Example 4 containing the phellodendron extract and the alteromonas ferment extract. It is assumed that this is caused by UV shielding effect of the phellodendron extract. It had been well known that phellodendron plant exhibits antifungal, antiinflammatory, and antivirus effects in skin. However, there had been no reports that the phellodendron plant can prevent discoloration and off-flavor by exposure to UV light. The present inventors found that the phellodendron plant can prevent phase instability of effective components in cosmetic compositions by sun light and the like.

### EXPERIMENTAL EXAMPLE 5

### Long-Term Storage Stability

To evaluate the long-term storage stability of the resveratrol used as the effective component in the stabilized compositions of the present invention, the compositions of Example 4 and Comparative Example 5 were stored in a water bath, which had been set to room temperature (R.T.), 40° C., and 4° C., for 3 months, and HPLC analysis for the resveratrol was performed. The test results of long-term storage stability for the resveratrol are summarized in Table 7 below.

TABLE 7
Long-term storage stability of resveratrol

| | Example 4 | | | Comparative Example 5 | | |
|---|---|---|---|---|---|---|
| Section | R.T. | 40° C. | 4° C. | R.T. | 40° C. | 4° C. |
| Initial | 100 | 100 | 100 | 100 | 100 | 100 |
| 1 month | 98.01 | 97.50 | 98.50 | 85.20 | 80.46 | 75.46 |
| 3 months | 95.20 | 94.30 | 96.40 | 76.45 | 70.25 | 65.25 |

HPLC analysis conditions for the long-term storage stability test of resveratrol were as follows:

1) Analysis column: Inertsil C8, 120A, Sum, 4.6×150 mm (GL science)

2) Mobile phase: mobile phase 1; 25 mM NaH₂PO₄, mobile phase 2; acetonitrile

TABLE 8
HPLC analysis

| Time (min) | Mobile phase 1 | Mobile phase 2 | Speed (ml/min) |
|---|---|---|---|
| 0 | 100 | 0 | 1.0 |
| 10.0 | 80 | 20 | 1.5 |
| 15.0 | 50 | 50 | 1.5 |
| 20.0 | 100 | 0 | 1.0 |
| 30.0 | 100 | 0 | 1.0 |

3) Column oven temperature: room temperature

4) Wavelength: 310 nm

5) Load amount: 10 μl

As shown in Table 8, the composition of Comparative Example 5, in which resveratrol was dissolved in a common solvent, exhibited poor long-term storage stability due to crystal precipitation at low temperature and phase instability at high temperature. On the other hand, the composition of Example 4, in which resveratrol was encapsulated with hydroxypropyl beta cyclodextrin, exhibited excellent long-term storage stability.

The test results for long-term storage stability of alpha-lipoic acid are summarized in Table 9 below.

TABLE 9
Long-term storage stability of alpha-lipoic acid

| | Example 4 | | | Comparative Example 5 | | |
|---|---|---|---|---|---|---|
| Section | R.T. | 40° C. | 4° C. | R.T. | 40° C. | 4° C. |
| Initial | 100 | 100 | 100 | 100 | 100 | 100 |
| 1 month | 97.10 | 96.40 | 98.10 | 83.90 | 80.46 | 85.60 |
| 3 months | 94.50 | 94.60 | 98.50 | 76.20 | 70.50 | 80.20 |

HPLC analysis conditions for the long-term storage stability test of alpha-lipoic acid were as follows:

1) Analysis column: Novapak C18, 120A, Sum, 4.6×150 mm (GL science)

2) Mobile phase: mobile phase 1; 0.1% trifluoroacetic acid (pH 2.4), mobile phase 2; acetonitrile

TABLE 10
HPLC analysis

| | | | Speed |
|---|---|---|---|
| Time (min) | Mobile phase 1 | Mobile phase 2 | (ml/min) |
| 0 | 100 | 0 | 1.0 |
| 3.0 | 100 | 0 | 1.0 |
| 8.0 | 50 | 50 | 1.0 |
| 13.0 | 50 | 50 | 1.0 |
| 20.0 | 100 | 0 | 1.0 |
| 25.0 | 100 | 0 | 1.0 |

3) Column oven temperature: room temperature

4) Wavelength: 333 nm

5) Load amount: 20 μl

As shown in Table 9, the composition of Comparative Example 5, in which alpha-lipoic acid was dissolved in a common solvent, exhibited poor long-term storage stability due to phase instability with increase of temperature. On the other hand, the composition of Example 4, in which alpha-lipoic acid was encapsulated with hydroxypropyl beta cyclodextrin, exhibited excellent long-term storage stability.

### EXPERIMENTAL EXAMPLE 6

### Skin Irritation Test

To evaluate skin safety, patch test in skins of healthy persons was performed using the compositions of Examples 4 and 5 and Comparative Examples 4 and 5 and the results are summarized in Table 11 below. Test method and evaluation method are as described in the above Experimental Example 4.

TABLE 11
Test results of skin irritation

| | Irritation | Degree of |
|---|---|---|
| Sample | score | irritation |
| Example 4 | 1.3 | Minimal |
| Example 5 | 2.1 | Mild |
| Comparative | 3.0 | Normal |
| Example 4 | | |
| Comparative | 4.5 | Severe |
| Example 5 | | |

As shown in Table 11, the compositions of Examples 4 and 5, in which a primary stabilizer of the present invention was used, exhibited more excellent skin safety. The composition of Example 5, in which the phellodenderon extract was used as a stabilizer, exhibited an irritation relief effect by excellent antiinflammatory activity, as compared to the composition of Comparative Example 4. In particular, the composition of Example 4, in which both the phellodendron extract and the alteromonas extract were used, exhibited more excellent skin irritation relief effect.

### EXPERIMENTAL EXAMPLE 7

### Long-Term Effect Test of Antioxidative Activity

To evaluate the long-term effects of the stabilized compositions of the present invention, a free radical scavenging activity test for the long-term effect of antioxidative activity of resveratrol and alpha-lipoic acid was performed and the results are summarized in Table 12 below.

The free radical scavenging activity test was performed as follows. Each sample of the compositions of Example 4, Comparative Examples 4 and 5 was collected at an initial time of preparation, 1 month and 3 months after the preparation. The collected sample was dissolved in 10 mg/ml of ethanol and, if necessary, diluted with ethanol, to obtain a sample solution. 1 ml of 0.1 mM solution of deep blue-colored DPPH(1,1-diphenyl-2-picryl-hydrazyl) radical in methanol was added to 1 ml of the sample solution, vigorously stirred for about 10 minutes, and incubated at 37° C. for 30 minutes. Then, the absorbance (ODₑₓₚ) of the resultant solution was measured at 516 nm by using spectrophotometer. Here, the degree of reduction of the absorbance was measured because the sample solution was changed from blue color to colorless as DPPH radical was scavenged. In addition, the sample solution containing no DPPH solution was used as control sample and the absorbance (OD_{con}) was measured. The absorbance (OD_{std}) of standard sample containing only DPPH solution was also measured.

Free radical scavenging rates were calculated from the above absorbance values. The concentrations for 50% free radical scavenging, i.e., IC₅₀ were calculated. The calculation method is as described in the above Experimental Example 2. As the value of IC₅₀ decreases, a free radical scavenging activity is excellent.

TABLE 12
Free radical scavenging activity

| | IC₅₀ (μg/ml) | | |
|---|---|---|---|
| Sample | Initial | 1 month | 3 months |
| Example 4 | 35 | 42 | 50 |
| Comparative Example 4 | 40 | 80 | >100 |
| Comparative | 48 | >100 | >1000 |
| Example 5 | | | |

As shown in Table 12, the composition of Example 4, in which the resveratrol and the alph-lipoic acid with excellent antioxidative activity but relatively poor long-term effect of antioxidative activity were stabilized by the primary stabilizer and the secondary stabilizer, exhibited an excellent long-term effect, as compared to the compositions of Comparative Examples 4 and 5.

### EXPERIMENTAL EXAMPLE 8

### Whitening Activity Test Based on Proliferation of Skin Melanocytes

To evaluate whitening activity, inhibitory activity of the compositions of Examples 7 and Comparative Examples 6-8 against melanin production in melanocytes of healthy persons were measured and the results are summarized in Table 13 below.

TABLE 13
Inhibitory activity of melanin production according
to the content of the water-soluble whitening composition

| Sample (concentration: | Inhibitory activity of melanin |
|---|---|
| 0.5%) | production (%) |
| Example 7 | 75.05 |
| Comparative Example 6 | 45.26 |
| Comparative Example 7 | 11.20 |
| Comparative Example 8 | 26.58 |

### EXAMPLE 8

### Preparation of Skin Toner

According to the composition ratios presented in Table 14 below, two components of a water part and five components of an alcohol part were respectively mixed with easy-mixer at room temperature. The alcohol part was gradually added to the water part and solubilized. After the addition of the alcohol part was completed, the reaction solution was further stirred for 10-20 minutes to obtain a skin toner.

TABLE 14
Skin toner

| Section | Component | Content | Content |
|---|---|---|---|
| Water part | Purified water | To 100 | To 100 |
| | Stabilized composition | 6.0 | — |
| | (Example 4) | | |
| Alcohol part | Ethanol | 6.0 | 6.0 |
| | Hydroxy castor oil | 1.0 | 1.0 |
| | POE (60) | | |
| | Flavor | Optimal | Optimal |
| | Preservative | Optimal | Optimal |
| | Dimethicone oil | 0.2 | 0.2 |

### EXAMPLE 9

### Preparation of Lotion

According to the composition ratios presented in Table 15 below, a water part containing five components and an oil part containing nine components were respectively heated at 75-80° C. until each component was completely dissolved. Then, the oil part was gradually added to the water part and primarily emulsified with homo-mixer at 75-80° C. at 3, 000 rpm for 5 minutes. During the primary emulsification, a neutralizer was added to the reaction mixture. After the primary emulsification was terminated, the resultant primary emulsion was cooled. Then, a flavor used as an additive was added at 50-55° C., secondarily emulsified with homo-mixer at 3,000 rpm for 3 minutes, and cooled to 27-30° C., to thereby obtain a lotion.

TABLE 15
Lotion

| Section | Component | Content |
|---|---|---|
| Water part | Purified water | To 100 |
| | Stabilized composition (Example 4) | 30 |
| | Preservative | Optimal |
| | Carbomer | 0.20 |
| | Xanthan gum | 0.05 |
| Oil part | Cetostearyl alcohol | 1.0 |
| | Self-emulsifiable glycerine | 1.0 |
| | monostearate | |
| | Sorbitan monostearate¹⁾ | 0.5 |
| | Glycerylmonostearate (POE40)²⁾ | 1.0 |
| | Liquid paraffin | 4.0 |
| | Squalane | 4.0 |
| | Cetyloctanoate | 6.0 |
| | Vasselin | 0.3 |
| | Beeswax | 0.3 |
| Neutralizer | Triethanolamine | 0.2 |
| Additive | Flavor | Optimal |

¹⁾Ar-60 (ICI, USA)
²⁾My-52 (ICI, USA)

### EXAMPLE 10

### Preparation of Cream

According to the composition ratios presented in Table 16 below, a water part containing five components and an oil part containing nine components were respectively heated at 75-80° C. until each component was completely dissolved. Then, the oil part was gradually added to the water part and primarily emulsified with homo-mixer at 75-80° C. at 3,000 rpm for 5 minutes. During the primary emulsification, a neutralizer was added to the reaction mixture. After the primary emulsification was terminated, the resultant primary emulsion was cooled. Then, a flavor used as an additive was added at 50-55° C., secondarily emulsified with homo-mixer at 3,000 rpm for 3 minutes, and cooled to 27-30° C., to thereby obtain a cream.

TABLE 16
Cream

| Section | Component | Content |
|---|---|---|
| Water part | Purified water | To 100 |
| | Stabilized composition (Example 4) | 40 |
| | Preservative | Optimal |
| | Carbomer | 0.30 |
| | Xanthan gum | 0.05 |
| Oil part | Cetostearyl alcohol | 1.5 |
| | Self-emulsifiable glycerine | 2.0 |
| | monostearate | |
| | Sorbitan monostearate¹⁾ | 1.0 |
| | Glycerylmonostearate (POE40)²⁾ | 1.5 |
| | Liquid paraffin | 6.0 |
| | Squalane | 5.0 |
| | Cetyloctanoate | 5.0 |
| | Vasselin | 0.5 |
| | Beeswax | 1.6 |
| Neutralizer | Triethanolamine | 0.3 |
| Additive | Flavor | Optimal |

¹⁾Ar-60 (ICI, USA)
²⁾My-52 (ICI, USA)

### EXAMPLE 11

### Preparation of Essence

According to the composition ratios presented in Table 17 below, a water part containing five components and an oil part containing five components were respectively heated at 75-80° C. until each component was completely dissolved. Then, the oil part was gradually added to the water part and primarily emulsified with homo-mixer at 75-80° C. at 3,000 rpm for 5 minutes. During the emulsification, a neutralizer was added to the reaction mixture. After the primary emulsification was terminated, the resultant primary emulsion was cooled. Then, a flavor used as an additive was added at 5 0-55° C., secondarily emulsified with homo-mixer at 3,000 rpm for 3 minutes, and cooled to 27-30° C., to thereby obtain an essence.

TABLE 17
Essence

| Section | Component | Content |
|---|---|---|
| Water part | Purified water | To 100 |
| | Stabilized composition (Example 4) | 45 |
| | Preservative | Optimal |
| | Carbomer | 0.40 |
| | Xanthan gum | 0.10 |
| Oil part | Cetostearyl alcohol | 1.20 |
| | Self-emulsifiable glycerine | 1.00 |
| | monostearate | |
| | Hydroxy castor oil POE (60) | 1.00 |
| | Cetyloctanoate | 5.00 |
| | Cyclomethicone | 3.00 |
| Neutralizer | Triethanolamine | 0.40 |
| Additive | Flavor | Optimal |

### EXPERIMENTAL EXAMPLE 9

### Whitening Activity Test in Human Bodies

The back portions of 20 healthy males/females (age 20 or more) were artificially blackened by a UV irradiator. Then, the appropriate amount of each of a test product and a control product was applied to the back portions of the test subjects, once a day for 6 weeks. The degree of white and black of skin was measured using chromameter (MINOLTA CR-300, Japan). Skin whitening activity was evaluated by measuring L, a, and b values of pigmented skin spots. In detail, skin whitening activity was evaluated by measuring L, a, and b values of the pigmented skin spots of the test subjects using Chromameter CR-300 at 0 weeks (0W), 1 week (1W), 2 weeks (2W), 3 weeks (3W), 4 weeks (4W), and 6 weeks (6W) after exposure to UV light (three times). Chromameter is an apparatus that represents colors using digital codes expressed by three parameters and is widely used to measure a skin color in the cosmetic industry [Muizzuddin N., Marenus K., Maes D., Smith W. P., Use of a chromameter in assessing the efficacy of anti-irritants and tanning accelerators, Journal of the society of Cosmetic Chemists, 1990; 41:369-378). In addition, the independent t-test (hypothesized mean difference: 5%) was performed to determine if there is a significance difference between the test product and the control product.

TABLE 18
Whitening activity in human bodies

| Section | Comparative Example 9 | Example 8 |
|---|---|---|
| 0W | 65.00 | 64.66 |
| 1W | 64.71 | 65.20 |
| 2W | 65.84 | 66.13 |
| 3W | 66.25 | 66.73 |
| 4W | 66.34 | 66.68 |
| 5W | 66.49 | 66.74 |
| 6W | 65.64 | 66.34 |
| P (T <= t) | | 0.028806 |
| Result value | 0.92 | 1.52 |

As shown in Table 18, the composition of Example 8 according to the present invention exhibited excellent whitening activity at a statistically significant level (p<0.05), as compared to the composition of Comparative Example 9.

### INDUSTRIAL APPLICABILITY

As apparent from the above description, a whitening and antioxidative cosmetic composition containing resveratrol of the present invention can provide excellent whitening and antioxidative effect even when used in small quantity. Furthermore, the cosmetic composition can stably contain the resveratrol. Even when it is stored for a long term, the cosmetic composition sustains excellent whitening and antioxidative effect without exhibiting problems such as discoloration, off-flavor, and crystal precipitation. In addition, the cosmetic composition exhibits a skin irritation relief effect.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A whitening and antioxidative cosmetic composition comprising resveratrol.

2. The whitening and antioxidative cosmetic composition of claim 1, wherein the resveratrol is used in an amount of 0.001-10.0 wt %, based on the total weight of the cosmetic composition.

3. The whitening and antioxidative cosmetic composition of claim 1, further comprising a primary stabilizer selected from the group consisting of cyclodextrin, polyethyleneglycol, and a mixture thereof.

4. The whitening and antioxidative cosmetic composition of claim 3, wherein the primary stabilizer is used in an amount of 0.1-50 wt %, based on the total weight of the cosmetic composition.

5. The whitening and antioxidative cosmetic composition of claim 3, further comprising a secondary stabilizer selected from the group consisting of an alpha-lipoic acid, a water-soluble whitening composition, a phellodendron extract, an alteromonas ferment extract, and a mixture thereof.

6. The whitening and antioxidative cosmetic composition of claim 5, wherein the secondary stabilizer is used in an amount of 0.01-15.0 wt %, based on the total weight of the cosmetic composition.

7. The whitening and antioxidative cosmetic composition of claim 5, wherein the water-soluble whitening composition is selected from the group consisting of a citrus unshiu peel extract, an aminoethylphosphinic acid, Waltheria indica extract, Guava Phenone, an aqueous hydrolyzed yeast solution, and a mixture thereof.

8. A method for preparing a whitening and antioxidative cosmetic composition, comprising:
(a) mixing a solvent and a primary stabilizer;
(b) adding resveratrol to the mixture of step (a); and
(c) adding a secondary stabilizer selected from the group consisting of an alpha-lipoic acid, a water-soluble whitening composition, a phellodendron extract, an alteromonas ferment extract, and a mixture thereof, to the mixture of step (b).
